(19) United States
(12) Patent Application Publication
(10) Pub. No.: US 2026/0124158 A1
(43) Pub. Date: May 7, 2026

(54) COMPOSITIONS AND METHODS TO REDUCE BIOLOGICAL AGE OF AN ADULT ANIMAL

(71) Applicant: Société des Produits Nestlé S.A., vevey (CH)

(72) Inventors: Gabriele CIVILETTO, Lausanne (CH); Giulia LIZZO, Echandens (CH); Philipp GUT, Genève (CH); Lorane TEXARI, Morges (CH)

(73) Assignee: Société des Produits Nestlé S.A., vevey (CH)

(21) Appl. No.: 19/117,945

(22) PCT Filed: Nov. 3, 2023
(86) PCT No.: PCT/EP2023/080626

§ 371 Date: Apr. 2, 2025

(30) Foreign Application Priority Data
Nov. 7, 2022 (EP) 22205779.6

(51) Int. Cl. A61K 31/05; A23K 20/111; A23K 50/40; A61P 43/00

(52) U.S. Cl. CPC A61K 31/05; A23K 20/111; A23K 50/40; A61P 43/00

(57) ABSTRACT

The present disclosure generally relates to compositions and methods to reduce the biological age and/or reduce the rate of increase of the biological age of an adult animal. More specifically, the present disclosure relates to administering a formulation comprising an effective amount of thymol and/or carvacrol, alone or in combination with a dietary regime such as caloric restriction, senior diet or low protein diet. The formulation can concomitantly reduce the biological age of a tissue, organ or physiological system in the adult animal in need; or treat, prevent, or delay the onset of an organ disease, or decline of organ function, or condition relating thereto in an adult animal in need.

### CROSS REFERENCE TO RELATED APPLICATIONS

This application claims priority to European application No. 22205779.6 filed Nov. 7, 2022 and PCT/EP 2023/080626 application filed Nov. 3, 2023, the disclosure of which is incorporated in its entirety herein by this reference.

### BACKGROUND TO THE INVENTION

The present disclosure generally relates to compositions and methods to reduce the biological age and/or reduce the rate of increase of the biological age of an adult animal. More specifically, the present disclosure relates to administering a formulation comprising an effective amount of thymol and/or carvacrol, alone or in combination with a dietary regime such as caloric restriction, senior diet or low protein diet. The formulation can concomitantly reduce the biological age of a tissue, organ or physiological system in the adult animal in need; or treat, prevent, or delay the onset of an organ disease, or decline of organ function, or condition relating thereto in an adult animal in need.

Chronological age is known to be a major indicator of general health status, with increasing chronological age associated with reduced health. However, depending on genetics, nutrition, and lifestyles, individuals may age slower or faster than their chronological age.

Chronological age may therefore not always reflect an individual's rate of aging or risk of reduced health. On the other hand, the biological age of an individual (based on e.g. clinical biochemistry and cell biology measures) can vary compared to others of the same chronological age.

Highly digestible and high-quality protein diets are generally recommended based upon the chronological age of a dog. For example, it may be recommended that a dog is switched to a senior diet around 7 or 8 years old. However, the determination of an increased biological age for a dog compared to its chronological age may allow a determination to provide additional nutritional solution to the animal.

There is thus a need for methods and compositions effective in reducing biological age and conditions related thereto, of an adult animal having an increased biological age compared to its chronological age.

### SUMMARY OF THE INVENTION

The inventors surprisingly found that thymol and/or carvacrol induces reduction in biological age at the individual level and at tissue level.

Accordingly, in a general embodiment, the present disclosure provides a method for reducing the biological age and/or reduce the rate of increase of the biological age of an adult animal in need, the method comprising: feeding the adult animal a composition comprising an effective amount of thymol and/or carvacrol.

The present disclosure also provides a method for reducing the biological age of a tissue, organ or physiological system in an adult animal in need, the method comprising feeding the adult animal a composition comprising an effective amount of thymol and/or carvacrol.

The present disclosure further provides a method for treating, preventing, or delaying the onset of an organ disease, or decline of organ function, or condition relating thereto in an adult animal in need, the method comprising feeding the adult animal a composition comprising an effective amount of thymol and/or carvacrol.

Suitably, the present methods may comprise selecting and/or applying the composition comprising an effective amount of thymol and/or carvacrol to an animal following a determination that the animal's biological age is greater than its chronological age. The increased biological age may be in a tissue, in an organ or in a physiological system such that immune, gastrointestinal, urinary, muscular, cardiovascular, and/or neurological system.

Suitably, improving the biological age of an animal may refer to a reduction in the difference between the biological age and chronological age of the animal, where the biological age of the animal is greater than its chronological age. Alternatively, a worsening in the biological age of an animal may refer to an increase in the difference between the biological age and chronological age of the animal, where the biological age of the animal is greater than its chronological age.

Suitably, improving the biological age of an animal may refer to a reduction in the rate of change between the biological age and chronological age of the animal, where the biological age of the animal is greater than its chronological age. For example, an animal's biological age may have been increasing by 1.5 years per 1 year increase in chronological age.

Suitably, whilst the present composition may be effective for animals based on chronological age, it may be particularly effective when applied to an animal with an increased biological age compared to its chronological age. As such, the present method may advantageously be beneficial for an animal that has an increased likelihood to respond, or improved magnitude of response, to the present composition.

The composition comprising thymol and/or carvacrol may be part of or administered together with, a dietary regime. The dietary regime may be a calorie-restricted diet, a senior diet or a low protein diet.

An advantage of one or more embodiments provided by the present disclosure is to improve the condition of animals, ageing animals, or ageing humans.

Still another advantage of one or more embodiments provided by the present disclosure is to increase the survivability of an ageing individual.

An additional advantage of one or more embodiments provided by the present disclosure is to increase mobility in the animal.

Additional features and advantages are described in, and will be apparent from, the following Detailed Description and the Figures.

### FIGURES

FIG. 1 shows the effects of age and thymol on biological age in muscle. Biological age has been estimated by analysis of DNA methylation using the pan tissue clock. Results are expressed as mean±S.E.M. and each data point represents a biological replicate. One-way ANOVA with post-hoc Dunnett's multiple comparison. ***P<0.001, *P<0.05

FIG. 2 shows the effects of age and thymol on biological age in muscle. Biological age has been estimated by analysis of DNA methylation using the intervention muscle clock. Results are expressed as mean±S.E.M. and each data point represents a biological replicate. One-way ANOVA with post-hoc Dunnett's multiple comparison. ***P<0.001, *P<0.05

### DETAILED DESCRIPTION

Various preferred features and embodiments of the present invention will now be described by way of non-limiting examples. The skilled person will understand that they can combine all features of the invention disclosed herein without departing from the scope of the invention as disclosed.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

Numeric ranges are inclusive of the numbers defining the range.

The publications discussed herein are provided solely for their disclosure prior to the filing date of the present application. Nothing herein is to be construed as an admission that such publications constitute prior art to the claims appended hereto.

### Subject

The present methods are directed to adult animals. Accordingly, the animal according to the present invention is a human, a pet animal, preferably a dog or a cat.

### Chronological Age

Chronological age may be defined as the amount of time that has passed from the subject's birth to the given date. Chronological age may be expressed in terms of years, months, days, etc. In certain embodiments, the animal is a dog and may be at least about 2 years old. Suitably, the dog may be at least about 2, at least about 3, at least about 4, at least about 5, at least about 6, at least about 7, at least about 8, at least about 9 or at least about 10 years old.

### Determination of the Biological Age

The present methods may comprise determining a DNA methylation profile to determine a biological age of an animal, e.g. a human or a dog using any suitable method.

A biological age for a particular tissue, organ, or physiological system may be determined using a DNA methylation profile comprising, or consisting of, methylation sites from genes that are preferentially or specifically expressed by that tissue, organ, or physiological system. Classifications of genes by a particular tissue, organ, or physiological system are publically available at, for example, Gene Ontology (http://geneontology.org/), the KEGG pathway database (https://www.genome.jp/kegg/), or MSIgDB (https://www.gsea-msigdb.org/gsea/msigdb/index.jsp).

The biological age of the dog may be expressed in terms of years, months, days, etc.

### Composition

The composition comprising an effective amount of thymol and or carvacrol, can be administered to an animal such as a human, e.g., an ageing individual or a pet animal, in a therapeutically effective dose. The therapeutically effective dose can be determined by the person skilled in the art and will depend on a number of factors known to those of skill in the art, such as the severity of the condition and the weight and general state of the individual.

Thymol (10-64%) is one of the major constituent of essential oils of thyme (Thymus vulgaris L., Lamiaceae). Carvacrol is present in the essential oil of Origanum vulgare (oregano), oil of thyme, oil obtained from pepperwort, and wild bergamot. The essential oil of thyme subspecies contains between 5% and 75% of carvacrol, while Satureja (savory) subspecies have a content between 1% and 45%. Origanum majorana (marjoram) and Dittany of Crete are rich in carvacrol, 50% and 60-80% respectively. Therefore, some embodiments of the composition comprise such plant and/or enriched plant extracts, essential oils or fractions that provide at least a portion of thymol and/carvacrol in the composition, in particular from thyme and oregano.

The composition can comprise a pharmacologically effective amount of thymol and/or carvacrol in a pharmaceutically suitable carrier. In aqueous liquid compositions, concentration preferably ranges from about 0.05 wt. % to about 4 wt. %, or from about 0.5 wt. % to about 2 wt. % or from about 1.0 wt. % to about 1.5 wt. % of the aqueous liquid composition.

In particular embodiments, the method is a treatment that augments the plasma thymol and/or carvacrol level in an individual, for example to a level in the range of 50 to 6000 nmol/L plasma, preferably 100 to 6000 nmol/L plasma. The method can comprise administering daily thymol and/or carvacrol in the weight range of 0.05 mg-1 g per kg body weight, preferably 1 mg-200 mg per kg body weight, more preferably 5 mg-150 mg per kg body weight, even more preferably 10 mg-120 mg per kg body weight, or most preferably 40 mg-80 mg per kg body weight.

Typically between 50 μg to 10 g of thymol and/or carvacrol, per daily serving in one or more portions is administered to an individual.

The composition is preferably administered to the individual at least two days per week, more preferably at least three days per week, most preferably all seven days of the week; for at least one week, at least one month, at least two months, at least three months, at least six months, or even longer. In some embodiments, the composition is administered to the individual consecutively for a number of days, for example at least until a therapeutic effect is achieved.

In an embodiment, the composition can be administered to the individual daily for at least 30, 60 or 90 consecutive days.

The above examples of administration do not require continuous daily administration with no interruptions. Instead, there may be some short breaks in the administration, such as a break of two to four days during the period of administration. The ideal duration of the administration of the composition can be determined by those of skill in the art.

In a preferred embodiment, the composition is administered to the individual orally or enterally (e.g. tube feeding). For example, the composition can be administered to the individual as a beverage, a capsule, a tablet, a powder or a suspension.

The composition can be any kind of composition that is suitable for human and/or animal consumption. For example, the composition may be selected from the group consisting of food compositions, dietary supplements, nutritional compositions, nutraceuticals, powdered nutritional products to be reconstituted in water or milk before consumption, food additives, medicaments, beverages and drinks. In an embodiment, the composition is an oral nutritional supplement (ONS), a complete nutritional formula, a pharmaceutical, a medical or a food product. In a preferred embodiment, the composition is administered to the individual as a beverage. The composition may be stored in a sachet as a powder and then suspended in a liquid such as water for use.

In an embodiment, the composition includes a source of protein. The composition can comprise a mixture of one or more types of protein, for example one or more (i) peptides, (ii) longer chains of amino acids, or (iii) free form amino acids; and the mixture is preferably formulated to achieve a desired amino acid profile/content.

At least a portion of the protein can be from animal or plant origin, for example dairy protein such as one or more of milk protein, e.g., milk protein concentrate or milk protein isolate; caseinates or casein, e.g., micellar casein concentrate or micellar casein isolate; or whey protein, e.g., whey protein concentrate or whey protein isolate. Additionally or alternatively, at least a portion of the protein can be plant protein such as one or more of soy protein or pea protein.

In an embodiment, the composition includes a source of carbohydrates. Any suitable carbohydrate may be used in the composition including, but not limited to, starch (e.g., modified starch, amylose starch, tapioca starch, corn starch), sucrose, lactose, glucose, fructose, corn syrup solids, maltodextrin, xylitol, sorbitol or combinations thereof.

The source of carbohydrates is preferably not greater than 50 energy % of the composition, more preferably not greater than 36 energy % of the composition, and most preferably not greater than 30 energy % of the composition. The composition can have a high protein:carbohydrate energy ratio, for example greater than 0.66, preferably greater than 0.9 and more preferably greater than 1.2.

In an embodiment, the composition includes a source of fat. The source of fat may include any suitable fat or fat mixture. Non-limiting examples of suitable fat sources include vegetable fat, such as olive oil, corn oil, sunflower oil, high-oleic sunflower, rapeseed oil, canola oil, hazelnut oil, soy oil, palm oil, coconut oil, blackcurrant seed oil, borage oil, lecithins, and the like, animal fats such as milk fat; or combinations thereof.

In a preferred embodiment, the composition further contains an autophagy inducer selected from the group consisting of urolithin (e.g., Urolithin A, B or D), rapamycin, Torin1, valproic acid, polyphenols (e.g., resveratrol), caffeine, metformin, 5′ AMP-activated protein kinase (AMPK) activators, L-type calcium channel inhibitors, and mixtures thereof. Non-limiting examples of suitable autophagy inducers are palmitic acid, 5-aminoimidazole-4-carboxamide riboside (AICAR), verapamil, nifedipine, diltiazem, piperazine phenothiazine derivatives (e.g., trifluoperazine), ketones (e.g., beta-hydroxybutyrate, ketone salts, or ketone ester derivatives) and mixtures thereof.

In some embodiments, the composition is administered to the individual in a single dosage form, i.e. all compounds are present in one product to be given to an individual in combination with a dietary regime.

The dietary regime may be a meal, a regime of meals, a supplement or a regime of supplements, or combinations of a meal and a supplement, or combinations of a meal and multiple supplements.

The dietary intervention or dietary product described herein may be any suitable dietary regime, for example, a calorie-restricted diet, a senior diet, a low protein diet, a phosphorous diet, low protein diet, potassium supplement diet, polyunsaturated fatty acids (PUFA) supplement diet, anti-oxidant supplement diet, a vitamin B supplement diet, liquid diet, selenium supplement diet, or diets supplemented with carnitine, branched chain amino acids or derivatives, or any combination of the above.

Suitably, the dietary regime or dietary product may be a calorie-restricted diet, a senior diet, or a low protein diet. Suitably, the dietary intervention or dietary product may be a calorie-restricted diet. Suitably, the dietary intervention or dietary product may be a low protein diet.

A dietary intervention may be determined based on the baseline maintenance energy requirement (MER) of the dog. Suitably, the MER may be the amount of food that stabilizes the dog's body weight (less than 5% change over three weeks).

By way of example, it is generally understood that younger, growing dogs benefit from a high energy/high protein diet; however, older dogs may have a lower energy requirement and therefore diets can be appropriately modified. In particular, many manufacturers produce a ‘senior’ range of dog food which is lower in calories, higher in fibre but has suitable levels of protein and fat for an older dog.

Suitably, a calorie-restricted diet may comprise about 50%, about 55%, about 60%, about 65%, about 75%, about 80%, about 85%, or about 90% of the dog's MER. Suitably, a calorie-restricted diet may comprise about 60% or about 75% of the dog's MER.

Suitably, a low-protein diet may comprise less than 20% protein (% dry matter). For example, a low-protein diet may comprise less than 19% (% dry matter).

The dietary regime may comprise a food, supplement and/or drink that comprises a nutrient and/or bioactive that mimics the benefits of caloric restriction (CR) without limiting daily caloric intake. For example, the food, supplement and/or drink may comprise a functional ingredient(s) having CR-like benefits. Suitably, the food, supplement and/or drink may comprise an autophagy inducer. Suitably, the food, supplement and/or drink may comprise fruit and/or nuts (or extracts thereof). Suitable examples include, but are not limited to, pomegranate, strawberries, blackberries, camu-camu, walnuts, chestnuts, pistachios, pecans. Suitably, the food, supplement and/or drink may comprise probiotics with or without fruit extracts or nut extracts.

The methods described herein can be useful for i) reducing the biological age and/or reduce the rate of increase of the biological age of an animal or ii) reducing the biological age of a tissue, organ or physiological system as well as iii) treating, preventing, or delaying onset of an organ disease or organ functional decline, or conditions relating thereto for any animal.

In an embodiment, the increased biological age may be in a tissue, in an organ or in a physiological system such that immune, gastrointestinal, urinary, muscular, cardiovascular, and/or neurological system. In a preferred embodiment, the tissue is skeletal muscle. Non-limiting examples of such muscle include one or more of the following: vastus lateralis, gastrocnemius, tibialis, soleus, extensor, digitorum longus (EDL), biceps femoris, semitendinosus, semimembranosus, gluteus maximus, extra-ocular muscles, face muscles or diaphragm.

Those skilled in the art will understand that they can freely combine all features of the present invention described herein, without departing from the scope of the invention as disclosed.

### EXAMPLES

The invention will now be further described by way of examples, which are meant to serve to assist the skilled person in carrying out the invention and are not intended in any way to limit the scope of the invention.

### Example 1

### Material and Methods

### Animal Care

All animal experiments comply with the International Guiding Principles for Biomedical Research Involving Animals developed by the Council for International Organizations of Medical Sciences and the following guidelines for animal welfare. 24 old mice aged 18 months and 12 young mice aged 6 months were kept in a temperature-and humidity-controlled environment with a 12:12-h light-dark cycle. Mice had access to nesting and enrichment materials and were provided with ad libitum access to water and food.

### Mice Treatment and Sample Collection

Old mice were split in two groups and received either 20 mg/kg body weight of thymol or vehicle by gavage daily for 12 weeks, whereas young mice received the vehicle only (n=12). Animals found dead or euthanized at the humane endpoint for pain or physical distress, have been excluded from the analysis. At the end of the treatment, mice were euthanized with isoflurane after 2-hours fasting. Muscle gastrocnemius has been dissected samples and snap frozen in liquid nitrogen and stored at −80° C.

### DNA Extraction

DNA extraction was performed on 100 mg of muscle using QIAGEN DNeasy Blood and Tissue kit (Cat. No: 69506) and following manufacturer recommendations. DNA was quantified using the DropSense technology (from Unchained Labs).

### Methylation Detection

Bisulfite conversion was performed on 500 ng of DNA using the Zymo EZDNA methylation kit (Cat. No: D5001) and following manufacturer recommendations. 200 ng of converted DNA was used to proceed to the Infinium HD methylation assay protocol following manufacturer recommendations. DNA methylation data were generated using custom arrays: HorvathMammalMethyl40 (Cat. No: 20027399) from Illumina with the consent of the Clock Foundation (Torrance, California), who developed the arrays. Arrays were scanned using the iScan from Illumina.

### Data Analysis

To calculate biological age, raw data have been analysed using algorithms (clocks) developed by the Clock Foundation. The following clocks for mouse species have been applied: a pan tissue clock, trained on the DNA methylation status of multiple tissues; an intervention muscle clock, trained on tissue-specific data from mice that underwent caloric restriction. All these clocks are based on methylation analysis of different CpGs and show robust chronological age predicting power. The results obtained for each sample corresponds to the biological age in years. Data were plotted and analysed using GraphPad Prism 9. Ordinary One-way ANOVA followed by Dunnett's multiple comparisons was performed to test the effect of age and thymol supplementation compared to their respective controls.

### Results

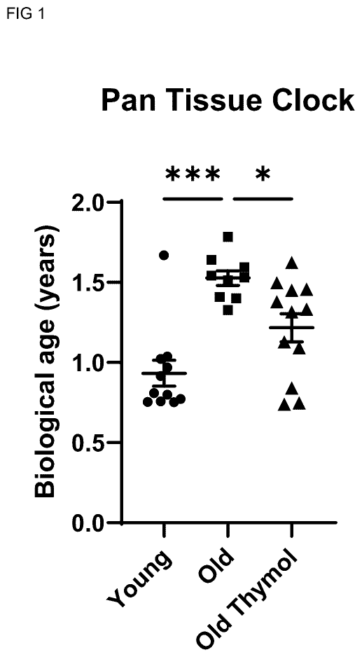

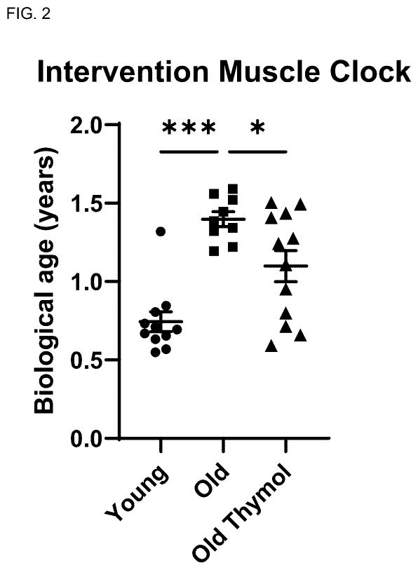

The results show that biological age increase in old mice (21 months old) compared to young mice (9 months old) as expected. Surprisingly, daily administration of thymol at the dose of 20 mg/kg/day for 12 weeks significantly reduced biological age in old mice. As shown in FIG. 1, treatment with thymol led to a reduction of 20.3% of muscle biological age measured with the pan tissue clock. As shown in FIG. 2 the effects of thymol on biological age were confirmed by using the interventions muscle clock, an algorithm that has been developed on interventions that delay aging, such as caloric restriction.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method for reducing the biological age and/or reduce the rate of increase of the biological age of an adult animal in need, the method comprising: feeding the adult animal a composition comprising an effective amount of thymol and/or carvacrol.

2. A method according to claim 1, wherein the feeding reduces the biological age of a tissue, organ or physiological system in the adult animal in need.

3. A method according to claim 2, wherein the tissue, organ or physiological system is immune, gastrointestinal, urinary, muscular, cardiovascular, and/or neurological system.

4. A method according to claim 2, wherein the tissue is skeletal muscle.

5. A method according to claim 1, wherein the feeding treats, prevents, or delays the onset of an organ disease, or decline of organ function, or condition relating thereto in the adult animal in need.

6. A method according to claim 1, wherein the animal in need has an increased biological age compared to its chronological age.

7. A method according to claim 6, wherein the increased biological age is determined from the DNA methylation profile of the adult animal.

8. A method according to claim 1, wherein the animal is an ageing animal.

9. A method according to claim 1, wherein the animal is a dog or a cat.

10. A method according to claim 1, wherein the animal is a human.

11. A method according to claim 1, wherein the composition is selected from the group consisting of food compositions, dietary supplements, nutritional compositions, nutraceuticals, powdered nutritional products to be reconstituted in water or milk before consumption, food additives, medicaments, drinks, dry or wet petfood and combinations thereof.

12. A method according to claim 1, wherein the composition is part of; or administered together with; a dietary regime selected from a calorie-restricted diet, a senior diet or a low protein diet.
